(19) United States
(12) Patent Application Publication
(10) Pub. No.: US 2006/0293721 A1
(43) Pub. Date: Dec. 28, 2006

(54) Vagus nerve stimulation for treatment of depression with therapeutically beneficial parameter settings

(75) Inventors: W. Brent Tarver, Houston, TX (US); Robert P. Cummins, Houston, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: Cyberonics, Inc., Houston, TX (US)

(21) Appl. No.: 11/191,321

(22) Filed: Jul. 28, 2005

Related U.S. Application Data
(60) Provisional application No. 60/694,535, filed on Jun. 28, 2005.

(51) Int. Cl. A61N 1/18

(52) U.S. Cl. 607/45

(57) ABSTRACT

Method and apparatus for treating a patient with depression comprising continuously providing a therapy to treat the patient's depression. The therapy, in one embodiment, comprises stimulating a patient's vagus nerve for about 30 seconds at a current of about 0.75 mA followed by a cessation of vagus nerve stimulation of about 5 minutes. Further still, the therapy comprises a pulse width of about 500 μs and a frequency of about 20 Hz. In another embodiment, the therapy comprises stimulating a patient's vagus nerve for about 25.07 seconds at a current of about 0.85 mA followed by a cessation of vagus nerve stimulation of about 4.07 minutes. This latter therapy also comprises a pulse width of about 415.20 μs and a frequency of about 20.07 Hz.

### BACKGROUND

1. Field of the Invention

The present subject matter relates generally to methods and apparatus for treating or controlling medical, psychiatric or neurological disorders by application of modulating electrical signals to a selected nerve or nerve bundle of the patient, and more particularly to techniques for treating patients with neuropsychiatric disorders by application of such signals to the vagus nerve, using an implantable neurostimulating device. Specifically, the invention is directed toward treating the symptoms of neuropsychiatric disorders such as schizophrenia, depression, and borderline personality disorder, by selective modulation of vagus nerve activity.

2. Description of Related Art

Schizophrenia was initially thought to have only psychological origins. Advances in psychobiology and psychopharmacology have revealed that the illness is primarily organic in nature. Electrophysiologic studies of patients with schizophrenia have supported an organic etiology. Although not entirely consistent, electroencephalogram (EEG) studies have tended to reveal abnormalities in these patients. Also, some parallels have been found between schizophrenia and epilepsy.

In Psych. Res. (1989) 29:419-420, Meuller reported finding increased beta (17.5 Hz) wave activity over the left central-temporal region during acute psychotic episode, whereas before and after the episode the frequency distribution in the EEG was normal. Williamson et al. in Can. J. Psych. (1989) 34:680-686, reported that a review of EEG mapping studies revealed that abnormalities exist, with some studies finding asymmetric fast activity while others reported primarily slowing. In Comprehensive Psych. (1990) 30(1):34-47, Keshaven et al. reported that sleep EEG studies in schizophrenic patients consistently showed abnormalities, and that although not specific to schizophrenia, patients tended to show impaired sleep continuity and reduced total sleep, but not all patients showed these abnormalities.

Gruzelier et al. reported in Int. J. Psychophysiol. (1990) 8:275-282, that in normal subjects the power of the beta II region of the EEG spectrum is decreased in cortical areas associated with specific mental tasks, this focal reduction in power being consistent with the thalamocortical EEG desynchronization response, and being decreased or absent in patients with schizophrenia. In Psychopathol. (1989) 22:65-140, Diehl indicated that acute psychotic episodes may be manifestations of temporal lobe epilepsy, and expressed the belief that disorders may exist in the ictal as well as the interictal phase. Kido et al. discussed six patients with seizures followed by schizophrenia-like states, in Japan J. Psych. Neurol. (1989) 43:433-438. In Intern. J. Neuroscience, Ardilla et al. described three cases in which patients diagnosed as psychotic were actually found to have complex partial status epilepticus.

Turning to depressive disorder, developments in psychobiology and psychopharmacology have provided considerable evidence that major depressive disorder and bipolar depression are biological rather than psychological diseases. Deficiency of brain neurostimulators has been associated with depression. In particular, abnormally low concentrations of serotonin and its metabolites have been found in depressed patients, as reviewed by Stark et al. in J. Clin. Psychopharmacol. (1985) 46[3, Sec.2]:7-13. Several serotonin uptake inhibitors, which increase the amount of serotonin at the synapse have been shown to be effective antidepressants. Serotonin is a neurotransmitter known to be involved in the brain stem projections of the vagus nerve in animals (Kilpatrick et al. in Eur. J. Pharmacol. (1989) 159:157-164) and in humans (Reynolds et al. Eur. J. Pharmacol. (1989) 174:127-130). It is postulated, then, that increased activity of the vagus nerve would be associated with release of more serotonin in the brain. The conclusion that depression has a biological basis is also supported by numerous electrophysiological and endocrine studies.

A paper by Pollock et al. in Biol. Psychiatry (1990) 27:757-780, reported that a review of studies of the EEG in awake depressed patients reveals that alpha and beta activity are increased compared to controls. Elevations of delta and theta frequency ranges were possibly present as well. It was also felt that increased beta activity may be particularly prominent in patients with coexistent anxiety. Buysee et al. reported in Arch. Gen. Psych. (1988) 45:568-575, finding that sleep EEG of patients with primary depression and secondary dementia showed a higher percentage of rapid eye movement (REM) and more phasic REM activity and intensity than patients with primary dementia and secondary depression.

A strong relationship has been found to exist between sleep and depression. One of the most effective treatments for depression is sleep deprivation, which, however, is not a practical long term therapy. As with schizophrenia, a relationship also appears to exist between depression and seizures.

A substantial body of data suggests that anti-convulsant compounds have a spectrum of therapeutic efficacy in a variety of psychiatric syndromes which have not been associated with an epileptoid process. Pathological degrees of neuronal excitability and/or dysregulation may be associated with marked alterations in behavior, which are potentially treatable with anticonvulsant compounds, even in the absence of a concurrent seizure disorder.

The use of electroconvulsive therapy (ECT) to induce seizures is a primary treatment in acute depressive disturbances. ECT appears equal or superior to traditional psychopharmacological treatment modes with tricyclic antidepressants. Although the precise mechanism by which the effect of ECT is achieved is not fully known, it is thought to be related to biochemical changes in the brain resulting from synchronous discharges associated with seizures. Antidepressant drugs may produce similar changes but without inducing seizures.

Certain anticonvulsant agents such as carbamazepine are used in psychiatric disorders. Some studies have indicated dramatic improvement by carbamazepine in affective and schizophrenia-like syndromes associated with epilepsy. Non-epileptic patients with nonspecific EEG abnormalities who suffer from marked psychiatric disorders have also been shown to respond favorably to this drug. In this group, improvements in violent behavior, irritability, emotional lability, depression, agitation, and apathy have been reported. Anticonvulsant compounds thus appear to have an important spectrum of clinical activity in neuropsychiatric syndromes in addition to their clinical utility in the treatment of epileptic disorders.

Borderline personality disorder is a poorly understood, but recognized psychiatric disorder which seems to have some overlap of schizophrenia and depression. Patients tend to be poorly functional without florid psychosis or overt depression. Lahmeyer et al reported, in J. Clin. Psych. (1989) 50(6):217-225, that sleep architecture in patients with borderline personality disorder is disturbed in that REM latency is decreased and REM density is increased. This was found to be particularly true if patients suffered coexisting depression, a history of affective illness or a family history of psychopathology. Sleep abnormalities were reported to appear similar to those seen in affective disorders.

In addressing a therapy involving nerve stimulation to treat such neuropsychiatric disorders, observation should be made of existing knowledge that most nerves in the human body are composed of thousands of fibers, having different sizes designated by groups A, B and C, carrying signals to and from the brain and other parts of the body. The vagus nerve, for example, may have approximately 100,000 fibers (axons) of the three different types, each of which carries such signals. Each axon of that nerve only conducts in one direction, in normal circumstances. The A and B fibers are myelinated, that is, they have a myelin sheath in the form of a substance largely composed of fat. On the other hand, the C fibers are unmyelinated.

Myelinated fibers are typically larger, have faster electrical conduction and much lower electrical stimulation thresholds than the unmyelinated fibers. Along with the relatively small amounts of electrical energy needed to stimulate the myelinated fibers, it is noteworthy that such fibers exhibit a particular strength-duration curve in response to a specific width and amplitude of stimulation pulse.

The A and B fibers are stimulated with relatively narrow pulse widths, from 50 to 200 microseconds (μs), for example. A fibers exhibit slightly faster electrical conductivities than the B fibers, and slightly lower electrical stimulation thresholds. The C fibers are relatively much smaller, conduct electrical signals very slowly, and have high stimulation thresholds typically requiring wider pulse widths (e.g., 300-1000 μs) and higher amplitudes for activation. Although the A and B fibers may be selectively stimulated without also stimulating the C fibers, the magnitude and width of the pulse required for stimulating the C fibers would also activate A and B fibers.

Although electrical stimulation of the nerve fiber typically activates neural signals in both directions (bidirectionally), selective unidirectional stimulation is achievable through the use of special nerve electrodes and stimulating waveforms. As noted above, each axon of the vagus nerve normally conducts in only one direction.

In a paper on the effects of vagal stimulation on experimentally induced seizures in rats (Epilepsia 1990, 31 (Supp 2): S7-S19), Woodbury has noted that the vagus nerve is composed of somatic and visceral afferents (i.e., inward conducting nerve fibers which convey impulses toward a nerve center such as the brain or spinal cord) and efferents (i.e., outward conducting nerve fibers which convey impulses to an effector to stimulate it and produce activity). The vast majority of vagal nerve fibers are C fibers, and a majority are visceral afferents having cell bodies lying in masses or ganglia in the neck. The central projections terminate, by and large, in the nucleus of the solitary tract which sends fibers to various regions of the brain (e.g., the hypothalamus, thalamus, and amygdala); others continue to the medial reticular formation of the medulla, the cerebellum, the nucleus cuneatus and other regions.

Woodbury further notes that stimulation of vagal nerve afferent fibers in animals evokes detectable changes of the EEG in all of these regions, and that the nature and extent of these EEG changes depends on the stimulation parameters. Chase, in Exp Neurol (1966) 16:36-49, had also observed that vagal activation can affect the EEG activity of certain parts of the brain. The applicants herein postulate that synchronization of the EEG may be produced when high frequency (>70 Hz) weak stimuli activate only the myelinated (A and B) nerve fibers, and that desynchronization of the EEG occurs when intensity of the stimulus is increased to a level that activates the unmyelinated (C) nerve fibers. Woodbury also observes that vagal stimulation can produce widespread inhibitory effects on seizures and certain involuntary movements.

Extra-physiologic electrical stimulation of the vagus nerve has previously been proposed for treatment of epilepsy and various forms of involuntary movement disorders. Specifically, in U.S. Pat. No. 4,702,254 issued Oct. 27, 1987 to J. Zabara (referred to herein as “the '254 patent”), a method and implantable device are disclosed for alleviating or preventing epileptic seizures, characterized by abnormal neural discharge patterns of the brain. The '254 patent describes an implantable neurocybernetic prosthesis (NCP) which utilizes neurocybernetic spectral discrimination by tuning the external current of the NCP generator to the electrochemical properties of a specific group of inhibitory nerves that affect the reticular system of the brain. These nerves are embedded within a bundle of other nerves, and are selectively activated directly or indirectly by the tuning of the NCP to augment states of brain neural discharge to control convulsions or seizures. According to the patent, the spectral discrimination analysis dictates that certain electrical parameters of the NCP pulse generator be selected based on the electrochemical properties of the nerves desired to be activated. The patent further indicates that the optimum sites for application of the NCP generator output to produce the desired effects are the cranial nerves in general, and the vagus nerve in particular.

The NCP disclosed in the '254 patent may be activated either manually or automatically, to provide treatment for the duration of the seizure. Manual activation is performed when the patient experiences the aura at onset of the seizure. Alternatively, automatic activation may be triggered upon detection of instantaneous changes in certain state parameters immediately preceding or at onset of a seizure. Additionally, a prophylactic or preventive mode may be employed in which the NCP is activated periodically to reduce the occurrence and/or the intensity of the seizures. The NCP stimulator of the '254 patent is implanted in the patient's chest and is connected to electrodes installed at the selected point of signal application at the nerve site with the more negative electrode situated closer to the brain and the positive electrode further from the brain, along the vagus nerve.

As for the treatment of any condition, it is desirable to provide an optimal therapy. Any improvements in the area of the treatment of neuropsychiatric disorders, such as depression, are desirable.

### SUMMARY OF THE INVENTION

In at least one embodiment of the invention, an implantable vagus nerve stimulator is provided to treat neuropsychiatric disorders (e.g., depression) wherein the stimulator is programmed in accordance with various configuration settings that have shown to produce therapeutically beneficial results. Methods are disclosed for treating a patient with depression comprising continuously providing a therapy to treat the patient's depression. The therapy, in one embodiment, comprises stimulating a patient's vagus nerve for about 30 seconds at a current of about 0.75 mA followed by a cessation of vagus nerve stimulation of about 5 minutes. Further still, the therapy comprises a pulse width of about 500 μs and a frequency of about 20 Hz. In another embodiment, the therapy comprises stimulating a patient's vagus nerve for about 25.07 seconds at a current of about 0.85 mA followed by a cessation of vagus nerve stimulation of about 4.07 minutes. This latter therapy also comprises a pulse width of about 415.20 μs and a frequency of about 20.07 Hz. Apparatus are also disclosed for providing the aforementioned therapies.

### BRIEF DESCRIPTION OF THE DRAWINGS

The above and still further objects, aspects, features and attendant advantages of the present invention will be better understood from a consideration of the ensuing detailed description of a presently preferred embodiment and method thereof, taken in conjunction with the accompanying drawings, in which:

FIG. 1 is a simplified block diagram of an implantable neurostimulator electronics package (stimulus generator) for use (with appropriate parameter settings and ranges) in treating neuropsychiatric disorders according to the present invention;

FIG. 2 is a simplified fragmentary illustration of a preferred embodiment of the stimulus generator and lead/electrode system of the neurostimulator implanted in the patient's body;

FIG. 3 is a detailed fragmentary illustration of the nerve electrode as implanted on the vagal nerve in the neck of the patient for modulating vagal activity;

FIG. 4 is an illustrative idealized electrical output signal waveform of the stimulus generator useful for clarifying relevant parameters of the signal developed by the stimulus generator for application to the nerve; and

FIG. 5 is a simplified block diagram of an EEG signal analysis circuit used in the stimulus generator.

### DETAILED DESCRIPTION OF THE PREFERRED EMBODIMENTS

Referring now to the drawings, a block diagram of the basic components of the stimulus generator of a neurostimulator and their interrelationship is illustrated in FIG. 1 and further details of location of an implantable version of the device and the associated lead/electrode system are shown in FIGS. 2 and 3. A generally suitable form of neurostimulator for use in the apparatus of the present invention is disclosed in U.S. Pat. No. 5,154,172, issued Oct. 13, 1992, to Anthony J. Varrichio et al., titled “Current Source with Programmable Overhead Voltage”, filed Nov. 10, 1989, and incorporated herein in its entirety by reference.

The neurostimulator utilizes a microprocessor and other electrical and electronic components, and in the case of an implanted device, communicates with a programmer and/or monitor located external to the patient's body by asynchronous serial communication for controlling or indicating states of the device. Passwords, handshakes and parity checks are employed for data integrity. The neurostimulator may also include means for conserving energy, which is desirable for any battery-operated medical device, and means for providing various safety functions such as preventing accidental reset of the device.

The stimulus generator 10 (FIG. 1) is preferably adapted to be implantable in the patient's body, in a pocket formed by the surgeon just below the skin in the chest as shown in FIG. 2, although a primarily external neurostimulator may alternatively be employed. The neurostimulator also includes implantable stimulating electrodes (described below) together with a lead system 22 for applying the output signal of the stimulus generator to the patient's vagus nerve. Components external to the patient's body include a programming wand for telemetry of parameter changes to the stimulus generator and monitoring signals from the generator, and a computer and associated software for adjustment of parameters and control of communication between the generator, the programming wand and the computer. The external components of the system are not shown in the drawings.

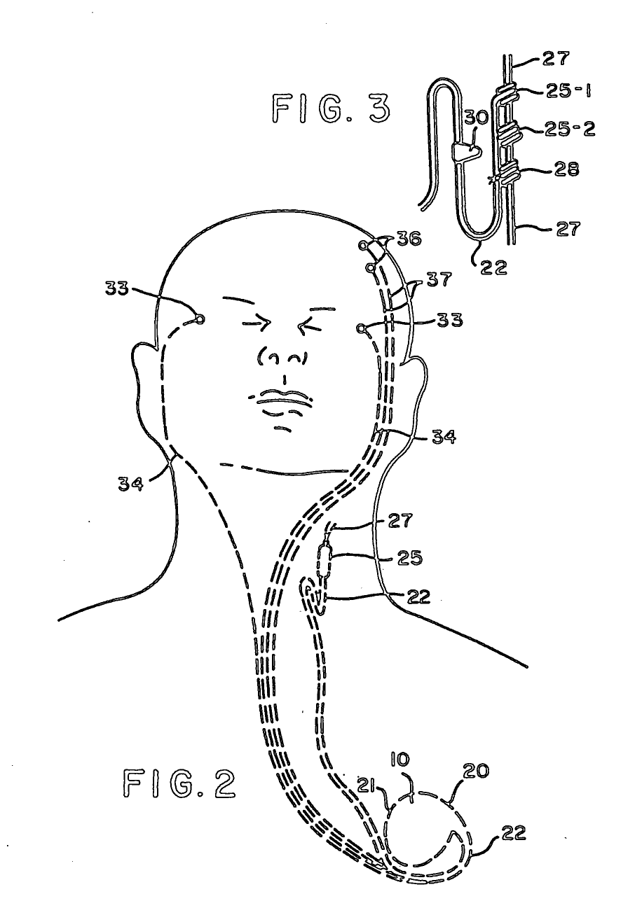

In conjunction with its microprocessor-based logic and control circuitry, the stimulus generator 10 or other implanted or external circuitry may include detection circuitry for sensing an event indicative of an abnormality to trigger automatic delivery of the stimulating signal. For example, surface or depth electrodes may be implanted to sense specific characteristics of the patient's EEG for triggering the therapy, as will be discussed presently in conjunction with the description of FIGS. 2 and 5. However, this involves complex and delicate electrode/lead implantation procedures as well as the requirement of circuitry for spectral analysis and/or programmable spectral or pattern recognition. Preferably, therefore, the treatment is applied continuously, periodically or intermittently or in accordance with the patient's circadian rhythm. The stimulus generator is designed, implemented and programmed to deliver a selectively patterned stimulating signal to modulate vagal activity in a manner designed to treat the specific neuropsychiatric disorder of interest.

As shown in FIG. 1, stimulus generator 10 includes a battery (or set of batteries) 12, which may be of any reliable long-lasting type conventionally employed for powering implantable medical electronic devices (such as batteries employed in implantable cardiac pacemakers or defibrillators). In the preferred embodiment of the stimulus generator, the battery is a single lithium thionyl chloride cell. The terminals of the cell 12 are connected to the input side of a voltage regulator 13. The regulator smoothes the battery output to produce a clean, steady output voltage, and provides enhancement thereof such as voltage multiplication or division if necessary for a specific application.

Regulator 13 supplies power to logic and control section 15, which includes a microprocessor and controls the programmable functions of the device. Among these programmable functions are output current, output signal frequency, output signal pulse width, output signal on-time, output signal off-time, daily treatment time for continuous or periodic modulation of vagal activity, and output signal-start delay time. Such programmability allows the output signal to be selectively crafted for application to the stimulating electrode set (FIGS. 2 and 3) to obtain the desired modulation of vagal activity for treatment and control of the disorder. Timing signals for the logic and control functions of the generator are provided by a crystal oscillator 16. A magnetically-actuated reed switch 14 may be incorporated in the electronics package to provide the generator with manual activation capability (by use of an external magnet, not shown, placed immediately adjacent to the package or its implant site).

Built-in antenna 17 enables communication between the implanted stimulus generator and the external electronics (including both programming and monitoring devices) to permit the device to receive programming signals for parameter changes, and to transmit telemetry information, from and to the programming wand. Once the system is programmed, it operates continuously at the programmed settings until they are reprogrammed (by the attending physician) by means of the external computer and the programming wand.

Logic and control section 15 of the stimulus generator 10 controls an output circuit or section 19 which generates the programmed signal levels appropriate to the disorder being treated. The output section and its programmed output signal are coupled (directly, capacitively, or inductively) to an electrical connector 20 on the housing 21 of the generator and to lead assembly 22 connected to the stimulating electrodes (FIGS. 2 and 3). If EEG sensing electrodes or eye movement sensing electrodes are to be implanted in the patient for triggering delivery of therapy by the stimulus generator on detection of an event indicative of the neuropsychiatric disorder of interest, a sense signal analysis circuit 23 is provided within the generator housing 21, with connections to the microprocessor in logic and control section 15 and to the sensing electrodes. An exemplary sense signal analysis circuit will be described presently.

Housing 21 in which stimulus generator 10 is encased is hermetically sealed and composed of a material such as titanium which is biologically compatible with the fluids and tissue of the patient's body. Further details of suitable structure and operation of the neurostimulator, beyond those by which the device is adapted to treat the neuropsychiatric disorder as described herein, are available in the '985. application, to which the reader is referred. Although not used in the preferred embodiment, if a detection system is employed with the neurostimulator to detect characteristics of the EEG, or to detect eye movement, by which to initiate the vagal stimulation automatically upon sensing the predetermined event indicative of need for treatment, the signal parameters of the implanted device may be calibrated by telemetry (via the programming wand) to the particular patient and the results then programmed into the microprocessor for the appropriate treatment.

FIG. 2 illustrates the preferred location of implanted generator 10, in case 21 with connector 20, in the patient's chest in a cavity formed by the implanting surgeon just below the skin, much as a pacemaker pulse generator would be implanted. A stimulating nerve electrode set 25 (FIG. 3) is conductively connected to the distal end of insulated electrically conductive lead assembly 22 which is attached at its proximal end to connector 20. Electrode set 25 is a bipolar stimulating electrode, preferably of the type described in U.S. Pat. No. 4,573,481 issued Mar. 4, 1986 to Bullara. The electrode assembly is surgically implanted on the vagus nerve 27 in the patient's neck. The two electrodes 25-1 and 25-2 are wrapped about the vagus nerve, and the assembly is secured to the nerve by a spiral anchoring tether 28 preferably as disclosed in U.S. Pat. No. 4,979,511 issued Dec. 25, 1990 to Reese S. Terry, Jr. and assigned to the same assignee as the instant application. Lead(s) 22 is secured, while retaining the ability to flex with movement of the chest and neck, by a suture connection 30 to nearby tissue.

The open helical design of electrode assembly 25 (described in detail in the above-cited Bullara patent), which is self-sizing and flexible, minimizes mechanical trauma to the nerve and allows body fluid interchange with the nerve. The electrode assembly conforms to the shape of the nerve, providing a low stimulation threshold by allowing a larger stimulation contact area. Structurally, the electrode assembly comprises two ribbons of platinum constituting the electrodes which are individually bonded to the inside surface of each of the first two spiral loops 25-1 and 25-2 of a three-loop helical assembly, and the two lead wires are respectively welded to the conductive ribbon electrodes. The remainder of each loop is composed of silicone rubber, and the third loop acts as the tether 28 for the electrode assembly. The inner diameter of the helical bipolar electrode assembly may typically be approximately two millimeters (mm), and an individual spiral is about seven mm long (measured along the axis of the nerve).

Eye movement sensing electrodes 33 may be implanted at or near the outer periphery of each eye socket in a suitable location to sense muscle movement or actual eye movement, as shown in FIG. 2, and electrically connected to leads 34 implanted via a catheter or other suitable means (not shown) and extending along the jawline through the neck and chest tissue to the sense signal analysis circuit 23 of stimulus generator 10. Sense electrodes 33 are utilized for rapid eye movement (REM) detection in a pattern indicative of the disorder to be treated, as will be described in greater detail below. Alternatively, or additionally, EEG sense electrodes 36 may be implanted in spaced apart relation through the skull, and connected to leads 37 implanted and extending along the scalp and temple and then along the same path and in the same manner as described above for the eye movement electrode leads. These or other types of sensing electrodes would only be required for alternative embodiments of the invention, since the preferred embodiment utilizes a continuous, periodic or intermittent stimulus signal applied to the vagus nerve (each of which constitutes a form of continual application of the signal), appropriate to treat the particular neuropsychiatric disorder which has been diagnosed in the case of the specific patient under observation.

The stimulus generator may be programmed with a computer using programming software of the type copyrighted by the assignee of the instant application with the Register of Copyrights, Library of Congress, or other suitable software based on the description herein, and a programming wand. An exemplary embodiment of an external programmer and wand and interaction between the programmer and the implanted device is shown in U.S. Pat. Nos. 5,707,400 and 6,473,644, incorporated herein by reference. The wand and software permit noninvasive communication with the generator after the latter is implanted. The wand is preferably powered by internal batteries, and provided with a “power on” light to indicate sufficient power for communication. Another indicator light is preferably provided to show that data transmission is occurring between the wand and the generator.

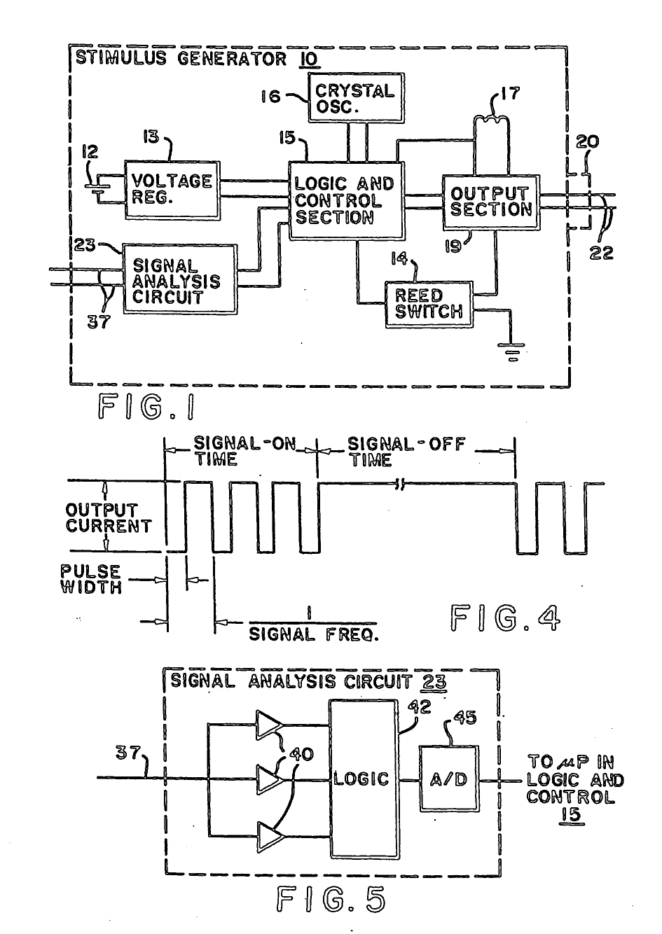

The operation of stimulus generator 10 to control and treat the neuropsychiatric disorder of interest will be described with reference to FIG. 4, which illustrates the general nature, in idealized representation, of the output signal waveform delivered by output section 19 of the neurostimulator to electrode assembly 25. This illustration is presented principally for the sake of clarifying terminology, including the parameters of output signal on-time, output signal off-time, output signal frequency, output signal pulse width, and output signal current.

In the treatment of schizophrenia according to the invention, the preferred stimulation strategy is to use circadian programming to desynchronize the EEG during the patient's normal waking hours, and to synchronize the EEG at night to improve sleep. Alternatively, detection strategies such as EEG detection of beta waves over the central temporal region, and/or of abnormal sleep patterns may be employed to trigger the stimulation. In the preferred embodiment and method, the vagal stimulation is continuously, periodically, or intermittently performed during prescribed segments of the patient's circadian cycle. For example, daytime stimulation may be periodic with a random frequency for the stimulating pulse waveform, with parameter selection for EEG desynchronization; and nighttime stimulation may employ a periodically applied pattern with parameters selected to synchronize the patient's EEG (e.g., at 90 Hz, 1 mA, 0.10 ms for the pulse waveform), alternating with desynchronizing stimuli at predetermined intervals (e.g., 100 minute separation) to produce low voltage fast (REM) activity. Such a regimen of vagal stimulation is programmed into the neurostimulator electronics package.

The schizophrenic patient is generally unable to recognize the symptoms of the disorder, and consequently no provision is made for patient activation of the neurostimulator for treatment of this particular disorder. However, the stimulus generator may be implemented for manual activation by a companion of the patient (using, for example, an external magnet to actuate the reed switch 14, in the implantable device of FIG. 1).

The preferred range of stimulation parameters for treatment of schizophrenia and the typical value of each parameter of the stimulating output signal are as follows:

| | | Desynch, | Synch, |
|---|---|---|---|
| | Range | Typical | Typical |
| Pulse Width | 0.05-1.5 ms | 0.5 ms | 0.1 ms |
| Output Current | 0.1-5.0 mA | 1.5 mA | 1.5 mA |
| Frequency | 5-150 Hz | 25 Hz | 80 Hz |
| On Time | 5-500 sec | 300 sec | 30 sec |
| Off Time | 5-500 sec | 10 sec | 5 sec |
| Frequency sweep | 10-50 Hz | Optional | Optional |
| Random frequency | 10-50 Hz | Optional | Optional |

Another activation modality for daytime stimulation is to program the output of the neurostimulator pulse generator to the maximum amplitude which the patient can tolerate, with cycling on and off for a predetermined period of time followed by a relatively long interval without stimulation.

For treating depression, a strategy is to employ circadian programming for night time stimulation to increase REM activity, and increase synchronization of the EEG during the patient's normal waking hours. Alternatively, a strategy may be employed for EEG detection of alpha or beta waveforms, and/or EEG detection and analysis of REM activity during sleep at night, followed by large signal, infrequent stimulation when the neurostimulator generator is activated by the detection circuitry. Here again, such detection may be implemented using surface or depth sensing electrodes and EEG spectral or REM analysis circuitry.

The patient suffering from depression is capable of recognizing the symptoms of the disorder, and therefore may be provided with a neurostimulator which is implemented, in the manner described above, to permit manual activation for delivery of the therapy. In the case of manual activation, the therapy applied preferably would be that normally employed during the patient's waking hours, i.e., to synchronize the EEG. It is unlikely, however, that an antidepressant effect would be achieved quickly, since treatment of depression using drugs begins to take effect in from two to four weeks and is probably related to changes in receptors, and the use of vagal stimulation for depression is likely to produce a similar result. For that reason, the neurostimulator should be programmed to generate the stimulus for a relatively long period of time in response to manual activation.

As noted earlier herein, the treatment is designed, in part, to increase the activity of the vagus nerve by which to evoke a release of greater amounts of the neurotransmitter serotonin in the patient's brain. This alteration, and specifically an increase, of the serotonin concentration in the brain is the result of an enhancement of the production of this natural antidepressant through vagal modulation.

A preferred range of stimulation parameters to treat depression, and the typical value of each parameter of the stimulus generator programmed output signal are as follows:

| | | Desynch, | Synch, |
|---|---|---|---|
| | Range | Typical | Typical |
| Pulse Width | 0.05-1.5 ms | 0.10 ms | 0.5 ms |
| Output Current | 0.1-5.0 mA | 1.0 mA | 1.5 mA |
| Frequency | 5-150 Hz | 90 Hz | 20 Hz |
| On Time | 5-500 sec | 30 sec | 300 sec |
| Off Time | 5-500 sec | 30 sec | 10 sec |
| Frequency sweep | 10-50 Hz | Optional | Optional |
| Random frequency | 10-50 Hz | Optional | Optional |

The circadian programming may also be set for synchronization of sleep patterns at night (e.g., output stimulating signal of 20 Hz, 500 ms, and 2 mA, cycled at 300 seconds on and 30 seconds off).

An activation modality for daytime stimulation in which the stimulus is applied to the nerve at the maximum amplitude tolerable by the patient, with on/off cycling for a first interval followed by a relatively long second interval without stimulation, similar to a modality described above for treating schizophrenia, may have value for treating depression. It bears some analogy to ECT which has been found effective in cases of depression, and would produce synchronous activity of the EEG for the brief stimulation intervals.

In the treatment of borderline personality disorder, the preferred stimulation strategy is designed to modify the patient's sleep patterns toward a normal pattern. Here, a suitable detection strategy is to employ implanted electrodes to sense muscle movement or actual eye movement during sleep, such as are shown in FIG. 2, and to analyze the detected REM activity; or to perform EEG detection with surface or depth EEG electrodes, followed by spectral analysis of the EEG. Again, however, circadian programming of the output signal for automatic stimulation in continuous, periodic or intermittent patterns is preferred for the sake of avoiding additional invasive procedures. In general, patient activation of the neurostimulation generator is not a viable option for the patient suffering from borderline personality disorder, although here again the provision of manual activation means could be appropriate for use by a companion.

The preferred range of stimulation parameters for treatment of borderline personality disorder and the typical value of each parameter of the programmed stimulation signal are as follows:

| | Range | Typical |
|---|---|---|
| Pulse Width | 0.05-1.5 ms | 0.10 ms |
| Output Current | 0.1-5.0 mA | 1.0 mA |
| Frequency | 5-150 Hz | 90 Hz |
| On Time | 5-1500 sec | 30 sec |
| Off Time | 5-1500 sec | 10 sec |
| Frequency sweep | 40-100 Hz | Optional |
| Random frequency | 40-100 Hz | Optional |

The circadian programming may employ specific patterns at night to modify REM activity for the purpose of increasing REM latency and to decrease REM intensity, tailored for each individual patient. Such a regimen of stimulation is best designed where the patient exhibits historically consistent sleep patterns, and would require defining the stimulation pattern for discrete time block during the sleep period.

In accordance with another embodiment of the invention, stimulus generator 10 is used to treat neuropsychiatric disorders (e.g., depression) by configuring the generator to a set of parameters that has shown to work well for statistically significant patient population. A study was conducted on a group of patients that have an implanted stimulus generator for the treatment of depression. Of the patients studied, 75 were considered to be “one year responders.” A responder is defined as a patient that experiences a 50%, or more, reduction in that patient's Hamilton Depression Rating Scale score after one year of treatment with an implanted stimulus generator 10 compared to that patient's score prior to implantation and treatment with the stimulus generator.

The following table provides the results of the 75, one-year responders. The results show median values of output current, pulse width, frequency, on-time and off-time of 0.75 mA, 500 μs, 20 Hz, 30 seconds, and 5 minutes, respectively. From the on-times and off-times, duty cycle can be calculated. The duty cycle for the average and median data is 9.3% and 9.09%, respectively.

Results from Depression patients, 1 year responders

| | Output | Pulse | | | |
|---|---|---|---|---|---|
| | Current | Width | Frequency | On-Time | Off-Time |
| Statistic | (mA) | (μs) | (Hz) | (sec) | (mm) |
| Average | 0.85 | 415.20 | 20.07 | 25.07 | 4.07 |
| (mean) | | | | | |
| Median | 0.75 | 500.00 | 20.00 | 30.00 | 5.00 |
| Mode | 0.50 | 500.00 | 20.00 | 30.00 | 5.00 |
| Max | 1.75 | 500.00 | 30.00 | 60.00 | 20.00 |
| Min | 0.00 | 130.00 | 10.00 | 7.00 | 0.30 |
| Standard | 0.66 | 147.82 | 5.06 | 10.36 | 8.48 |
| deviation | | | | | |

In some embodiments, the generator 10 is programmed at “about” the average or median values. In at least some embodiments, the term “about” refers to one standard deviation (shown in the table below) with reference to the average values. That is, an output current of “about” 0.85 mA comprises a current in the range of 0.19 mA to 1.51 mA, inclusive (assuming a standard deviation as shown of 0.66). Similar calculated ranges for the pulse width, frequency, on-time and off-time can also be made with the average and standard deviation values shown in the table above. In other embodiments, “about” refers to +/−5% or +/−10% with respect to the average or median values. Further still, with regard to output current, pulse width, frequency, on-time, and off-time, “about” refers to +/−0.25 mA, +/−50 μs or 100 μs, +1-5 Hz, +/−10 second, and +/−1 minute, respectively. Further still, again with regard to output current, pulse width, frequency, on-time, and off-time, “about” refers to the nearest quarter mA increment, the nearest 50 μs or 100 μs increment, the nearest 5 Hz increment, the nearest 10 second increment, and the nearest 1 minute increment, respectively.

Thus, the pulse generator permits the treatment of a patient with depression by continuously providing a therapy to treat the patient's depression. The therapy, in one embodiment, comprises stimulating a patient's vagus nerve for about 30 seconds at a current of about 0.75 mA followed by a cessation of vagus nerve stimulation of about 5 minutes. Further still, the therapy comprises a pulse width of about 500 μs and a frequency of about 20 Hz. In another embodiment, the therapy comprises stimulating a patient's vagus nerve for about 25.07 seconds at a current of about 0.85 mA followed by a cessation of vagus nerve stimulation of about 4.07 minutes. This latter therapy also comprises a pulse width of about 415.20 μs and a frequency of about 20.07 Hz.

If sense electrodes are to be utilized to detect onset of the disorder being treated, the signal analysis circuit 23 is incorporated in the stimulus generator 10 (FIG. 1).

Referring to FIG. 5, where the sense electrodes are EEG electrodes such as 36 and associated leads 37 of FIG. 2, analysis circuit 23 is implemented for EEG detection and analysis. To that end, circuit 23 includes a plurality of parallel active sense signal bandpass filters 40 staged to provide selective filtering in the ranges from 0-2 Hz, 2-4 Hz and 15-20 Hz, for example; a logic circuit 42 to select the output of one filter from among the plurality of filters 40; and an analog/digital (A/D) converter 45. The outputs of the filters are individually sampled by the logic circuit 42, and the sampling rate, averaging time interval, and weighting assigned to each sense signal band, are controlled by the microprocessor in the logic and control section 15 of the stimulus generator 10 (FIG. 1), to detect the EEG pattern. Upon detection of the symptom of interest of the disorder being treated, the processed digital signal is supplied to the microprocessor to trigger application of the stimulating signal to the patient's vagus nerve.

The activation of the analysis circuit 23 and its internal component circuitry need not be continuous, but only periodic such as every few hours, depending on the disorder being treated.

Various features may be incorporated into the neurostimulator for purposes of the safety and comfort of the patient. For example, comfort would be enhanced by programming the output stimulus to ramp up during the first two seconds of stimulation, rather than to be delivered abruptly. Also, the implanted generator may be provided with a clamping circuit to limit the maximum voltage, to 14 volts for example, which is delivered to the vagus nerve. Such a maximum limit is designed to prevent damage to the patient's vagus nerve.

The programmable functions and capabilities of the neurostimulator are designed and implemented to permit noninvasive communication with the stimulus generator after it is implanted, which is useful for both activation and monitoring functions. Beyond the essential functions of the device, the programming software may readily be structured to provide straightforward menu-driven operation, HELP functions, prompts, and messages to facilitate simple and rapid programming while keeping the user fully informed of everything occurring at each step of a sequence. Programming capabilities should include capability to modify the adjustable parameters of the stimulus generator and its output signal, to test device diagnostics, and to store and retrieve telemetered data. It is desirable that when the implanted unit is interrogated, the present state of the adjustable parameters is displayed on the monitor of external PC so that the programmer may then conveniently change any or all of those parameters at the same time; and, if a particular parameter is selected for change, all permissible values for that parameter are displayed so that the programmer may select an appropriate desired value for entry into the neurostimulator.

Diagnostics testing should be implemented to verify proper operation of the device, and to indicate the existence of problems such as with communication, the battery, or the lead/electrode impedance. A low battery reading, for example, would be indicative of imminent end of life of the battery and need for implantation of a new device. The nerve electrodes are capable of indefinite use absent indication of a problem with them observed on the diagnostics testing.

Although a preferred embodiment of apparatus and certain preferred methods for treating and controlling neuropsychiatric disorders through vagal modulation according to the invention have been described herein, it will be apparent to those skilled in the field from a consideration of the foregoing description that variations and modifications of such embodiments, methods and techniques may be made without departing from the true spirit and scope of the invention. For example, although a totally implantable device is preferred, the electronic energization package may, if desired, be primarily external to the body. Stimulation can be achieved with an RF power device implemented to provide the necessary energy level. The implanted components may be limited to the lead/electrode assembly, a coil and a DC rectifier. Pulses programmed with the desired parameters would be transmitted through the skin with an RF carrier, and the signal thereafter rectified to regenerate a pulsed signal for application as the stimulus to the vagus nerve to modulate vagal activity. This would virtually eliminate the need for battery changes. The disadvantages of such an implementation are that the external transmitter must be carried by the patient, greater power is required for activation, and the output current to the nerve is less stable.

An external stimulus generator may be employed with leads extending percutaneously to the implanted nerve electrode set. The major problem encountered with this technique is the potential for infection, but it is useful to allow short term testing of the patient to determine whether the particular neuropsychiatric disorder suffered by the patient under observation is amenable to successful treatment. If it is, a more permanent implant may be provided.

Accordingly, it is intended that the invention shall be limited only to the extent required by the appended claims and the rules and principles of applicable law.

What is claimed is:

1. A method of treating a patient with depression, comprises:
continuously providing a therapy to treat the patient's depression, said therapy comprising stimulating a patient's vagus nerve for about 30 seconds at a current of about 0.75 mA followed by a cessation of vagus nerve stimulation of about 5 minutes.

2. The method of claim 1 wherein said therapy also comprises, during the 30 seconds of stimulation, stimulating the patient's vagus nerve at a pulse width of about 500 μs.

3. The method of claim 2 wherein said therapy also comprises, during the 30 seconds of stimulation, stimulating the patient's vagus nerve at a frequency of about 20 Hz.

4. The method of claim 1 wherein said therapy also comprises, during the 30 seconds of stimulation, stimulating the patient's vagus nerve at a frequency of about 20 Hz.

5. The method of claim 1 wherein about 0.75 mA comprises a range selected from a group consisting of 0.75 mA +/−5%, 0.75 mA +/−10%, and 0.5 mA to 1 mA.

6. A method of treating a patient with depression, comprises:
continuously providing a therapy to treat the patient's depression, said therapy comprising stimulating a patient's vagus nerve for about 25.07 seconds at a current of about 0.85 mA followed by a cessation of vagus nerve stimulation of about 4.07 minutes.

7. The method of claim 6 wherein said therapy also comprises, during the 25.07 seconds of stimulation, stimulating the patient's vagus nerve at a pulse width of about 415.20 μs.

8. The method of claim 7 wherein said therapy also comprises, during the 25.07 seconds of stimulation, stimulating the patient's vagus nerve at a frequency of about 20.07 Hz.

9. The method of claim 6 wherein said therapy also comprises, during the 25.07 seconds of stimulation, stimulating the patient's vagus nerve at a frequency of about 20.07 Hz.

10. The method of claim 6 wherein about 0.85 mA comprises a range selected from a group consisting of 0.85 mA +/−5%, 0.85 mA +/−10%, 0.6 mA to 1.1 mA, and 0.75 to 1 mA.

11. An implantable pulse generator, comprising:
an output section adapted to couple to a plurality of electrodes;
a logic and control section coupled to the output section, wherein said logic and control section continuously causes a therapy to be provided to treat the patient's depression, said therapy comprising stimulating a patient's vagus nerve for about 30 seconds at a current of about 0.75 mA followed by a cessation of vagus nerve stimulation of about 5 minutes.

12. The pulse generator of claim 11 wherein said therapy also comprises, during the 30 seconds of stimulation, stimulating the patient's vagus nerve at a pulse width of about 500 μs.

13. The pulse generator of claim 12 wherein said therapy also comprises, during the 30 seconds of stimulation, stimulating the patient's vagus nerve at a frequency of about 20 Hz.

14. The pulse generator of claim 11 wherein said therapy also comprises, during the 30 seconds of stimulation, stimulating the patient's vagus nerve at a frequency of about 20 Hz.

15. The pulse generator of claim 11 wherein about 0.75 mA comprises a range selected from a group consisting of 0.75 mA +/−5%, 0.75 mA +/−10%, and 0.5 mA to 1 mA.

16. An implantable pulse generator, comprising:
an output section adapted to couple to a plurality of electrodes;
a logic and control section coupled to the output section, wherein said logic and control section continuously causes a therapy to be provided to treat the patient's depression, said therapy comprising stimulating a patient's vagus nerve for about 25.07 seconds at a current of about 0.85 mA followed by a cessation of vagus nerve stimulation of about 4.07 minutes.

17. The pulse generator of claim 16 wherein said therapy also comprises, during the 25.07 seconds of stimulation, stimulating the patient's vagus nerve at a pulse width of about 415.20 μs.

18. The pulse generator of claim 17 wherein said therapy also comprises, during the 25.07 seconds of stimulation, stimulating the patient's vagus nerve at a frequency of about 20.07 Hz.

19. The pulse generator of claim 16 wherein said therapy also comprises, during the 25.07 seconds of stimulation, stimulating the patient's vagus nerve at a frequency of about 20.07 Hz.

20. The pulse generator of claim 16 wherein about 0.85 mA comprises a range selected from a group consisting of 0.85 mA +/−5%, 0.85 mA +/−10%, 0.6 mA to 1.1 mA, and 0.75 to 1 mA.
